(19) United States
(12) Patent Application Publication
(10) Pub. No.: US 2024/0215904 A1
(43) Pub. Date: Jul. 4, 2024

(54) Ovulation Monitoring Platform

(71) Applicant: University of Cincinnati, Cincinnati, OH (US)

(72) Inventor: Jason Heikenfeld, Cincinnati, OH (US)

(73) Assignee: University of Cincinnati, Cincinnati, OH (US)

(21) Appl. No.: 18/684,137

(22) PCT Filed: Aug. 23, 2022
(86) PCT No.: PCT/US22/41183

§ 371 Date: Feb. 15, 2024

Related U.S. Application Data
(60) Provisional application No. 63/235,931, filed on Aug. 23, 2021.

(51) Int. Cl. A61B 5/00; A61B 5/145; A61B 5/1473

(52) U.S. Cl. CPC A61B 5/4306; A61B 5/1451; A61B 5/1473

(57) ABSTRACT

A device for measuring one or more analytes in a sample of interstitial fluid or blood is provided. The device comprises at least one EAB sensor using one or more attached redox couples that measure at least one of the analytes. It also includes at least one means to establish fluid communication between the sensor and the sample. The analytes are selected from the group consisting of progesterone, luteinizing hormone (LH), estrogen, follicle stimulating hormone (FSH), their metabolites, and combinations thereof.

### CROSS REFERENCE TO RELATED APPLICATIONS

This application is a continuation of PCT Application No. PCT/US22/41118 filed Aug. 22, 2022, which claims benefit of the filing date of U.S. Provisional Application Ser. No. 63/235,931, filed Aug. 23, 2021, the disclosures of which are incorporated by reference herein in their entireties.

### TECHNICAL FIELD

The present invention relates to devices and methods for monitoring biofluids related to ovulation.

### BACKGROUND OF THE INVENTION

Ovulation monitoring is a commercially proven technique utilized by women that is currently able to improve the rate of achieving pregnancy from 30% to 43%, enabled by a lateral flow assay that non-quantitatively measures in urine the onset of a surge of a protein called luteinizing hormone (LH). Although improving pregnancy rates, monitoring the LH surge in urine samples is actually a poor predictor of ovulation, given that 19% of women ovulate before their urine-based LH test turns positive. The actual trigger for ovulation is when serum LH levels reach a certain threshold. Between the time it takes for high serum LH levels to reach the urine and the infrequency women monitor their urine for LH, many women miss there most fertile time to engage in intercourse to result in conception. Therefore, urine LH testing has major limitations. Post-ovulation progesterone monitoring is also of value to ovulation monitoring. It is currently limited to progesterone metabolite monitoring in urine. Like LH monitoring in urine, progesterone monitoring requires multiple tests to capture a progesterone rise and peak in the body. Although the peak measurement of progesterone is not as important as peak measurement of LH, none-the-less, having to perform multiday measurements puts a testing burden on the women who may use such tests.

Interstitial fluid contains many of the same analytes as blood and often at comparable concentrations. As a result, interstitial fluid presents an alternative biofluid to blood or urine for detection of analytes such as LH. Commonly employed practices for continuous monitoring of glucose in interstitial fluid include in-dwelling sensors, where a needle is utilized to insert the sensor into the dermis of the skin, and micro-needles where the sensor is placed ex-vivo and the analyte is coupled from interstitial fluid to the sensor by diffusion to the sensor. However, no such capability exists for LH or progesterone, relegating clinical investigation of LH and progesterone to repeated in-clinic blood draws from the woman.

### SUMMARY OF THE INVENTION

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings LH and progesterone sensing technology into proximity with biofluids such as interstitial fluid.

In one embodiment, the present invention is a device for measuring one or more analytes in a sample of interstitial fluid, blood or both. The device comprises at least one electrochemical aptamer-based (EAB) sensor using one or more attached redox couples that measure at least one of said analytes; and at least one means to establish fluid communication between the at least one sensor and the sample of interstitial fluid, blood or both. The one or more analytes are selected from the group consisting of progesterone, luteinizing hormone (LH), estrogen, follicle stimulating hormone (FSH), their metabolites, and combinations thereof. In one embodiment, the means to establish fluid communication is at least one needle. In another embodiment, the needle is hollow and couples interstitial fluid or blood to the at least one sensor by diffusion. In another embodiment, the needle is hollow and couples interstitial fluid or blood to the at least one sensor by advection.

In another embodiment, the device of the present invention is capable of providing continuous measurement for at least 6 hours. In one embodiment, the device of the present invention is capable of providing continuous measurement for at least 24 hours. In another embodiment, the device of the present invention can provide a single measurement. The device of the present invention, in one embodiment, further incorporates a first sensor and a second sensor, and the first sensor measures LH and the second sensor measures progesterone.

In another embodiment of the present invention, a method of predicting and/or confirming ovulation of a subject is provided. The method involves applying a device for measuring one or more analytes from interstitial fluid, blood or both of the subject; and measuring a concentration change of luteinizing hormone (LH), progesterone or both in the subject. The one or more analytes are selected from the group consisting of progesterone, luteinizing hormone (LH), estrogen, follicle stimulating hormone (FSH), their metabolites, and combinations thereof. Further, the device includes at least one electrochemical aptamer sensor comprising one or more attached redox couples that measure at least one of said analytes; and at least one means to establish fluid communication between the at least one sensor to interstitial fluid, blood or both.

In one embodiment, the method further includes the use of at least two urine-based LH test strips to indicate a time for use of the device, and the device continuously measures at least LH for at least 6 hours. In another embodiment, the device continuously measures at least LH for at least 24 hours.

In another embodiment, the method also involves taking at least one measurement of progesterone by a device containing a sensor for progesterone. In one embodiment, the device contains sensors for both LH and progesterone and measures them both continuously for at least 6 hours. In another embodiment, the device reports to the user data selected from the group consisting of continuous concentration data of at least one of said analytes, a predicted time of ovulation, a predicted time of peak fertility, a confirmation that ovulation has occurred, and combinations thereof.

### BRIEF DESCRIPTION OF THE DRAWINGS

The objects and advantages of the disclosed invention will be further appreciated in light of the following detailed descriptions and drawings in which:

FIG. 1A is a representation of an aptamer sensing element.

FIG. 1B is a representation of an aptamer sensing element.

FIG. 2A is a cross-sectional view of a device according to an embodiment of the disclosed invention.

FIG. 2B is a cross-sectional view of a device according to another embodiment of the disclosed invention.

FIG. 3 is a graph showing an example plot of hormone levels during a subject's ovulation cycle.

### DEFINITIONS

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “analyte” means an oligonucleotide or polynucleotide having a sequence to which a particular electrode-bound oligonucleotide is designed to hybridize. It can also refer to a small molecule of the like to which a particular aptamer is designed to hybridize.

As used herein, the term “aptamer” means any polynucleotide molecule (for example, DNA or RNA molecule containing natural or synthetic nucleotides) that has the ability to bind other molecules. For example, aptamers have been selected which bind nucleic acids, proteins, small organic components and even entire organisms.

The particular use of terms “oligonucleotide” and “polynucleotide” should in no way be considered limiting. “Oligonucleotide” is used when the relevant nucleic acid molecules typically comprise less than about 100 bases. “Polynucleotide” is used when the relevant nucleic acid molecules typically comprise more than about 100 bases. Both terms are used to denote DNA, RNA, modified or synthetic DNA or RNA (including but not limited to nucleic acids comprising synthetic and naturally-occurring base analogs, dideoxy or other sugars, and thiols), and PNA or other nucleobase containing polymers. However, probes and/or targets may comprise fewer than or more than 100 bases (inclusive). Accordingly, the terms “oligonucleotide” and “polynucleotide” are used to describe particular embodiments of the invention. The terms in no way define or limit the length of the nucleic acids that may be used to practice the invention.

As used herein, “biofluid” may mean any human biofluid, including, without limitation, sweat, interstitial fluid, blood, plasma, serum, tears, and saliva.

As used herein, “continuous sensing” with a “continuous sensor” means a sensor that reversibly changes in response to concentration of an analyte, where the only requirement to increase or decrease the signal of the sensor is to change the concentration of the analyte in the biofluid. Such a sensor, therefore, does not require regeneration of the sensor by locally changing pH, for example. Similarly, as used herein, “continuous monitoring or measurement or sensing” means the capability of a device to provide multiple measurements of an analyte over time.

“EAB sensor” means an electrochemical aptamer-based biosensor that is configured with multiple aptamer sensing elements that, in the presence of a target analyte in a fluid sample, produce a signal indicating analyte capture, and which signal can be added to the signals of other such sensing elements, so that a signal threshold may be reached that indicates the presence or concentration of the target analyte. As non-limiting examples, such sensors can be in the forms disclosed in U.S. Pat. Nos. 7,803,542 and 8,003,374.

As used herein, “fluid communication” means that fluid can flow from one component to another; such flow may be by way of one or more intermediate (and not specifically mentioned) other components; and such may or may not be selectively interrupted (e.g., with a valve).

As used herein, the expression “interstitial fluid” means the substantially clear, substantially colorless fluid found in the human body, which occupies the space between the cells of the human body.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The present invention involves novel devices and methods for monitoring biofluids related to ovulation. In one embodiment, the device of the present invention uses a needle or microneedles connected to one or more EAB sensors to monitor biofluid analytes related to ovulation. Such analytes include progesterone, luteinizing hormone (LH), estrogen, follicle stimulating hormone (FSH), their metabolites, and combinations thereof. In one embodiment, the device of the present invention is capable of continuous sensing of these analytes over a certain period of time. Some embodiments of the disclosed invention are directed to continuous aptamer-based sensors for LH or progesterone in interstitial fluid or other biofluids.

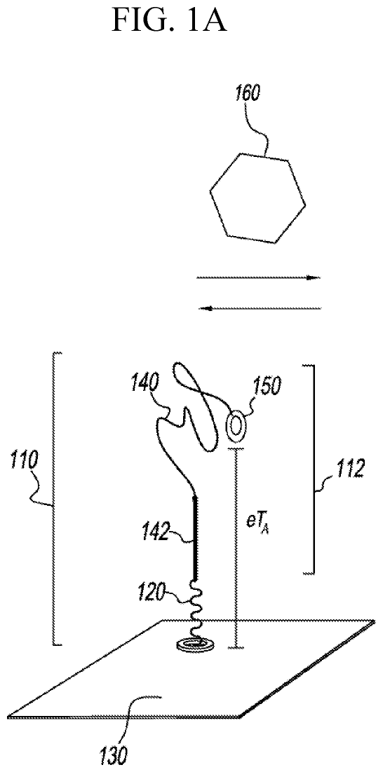

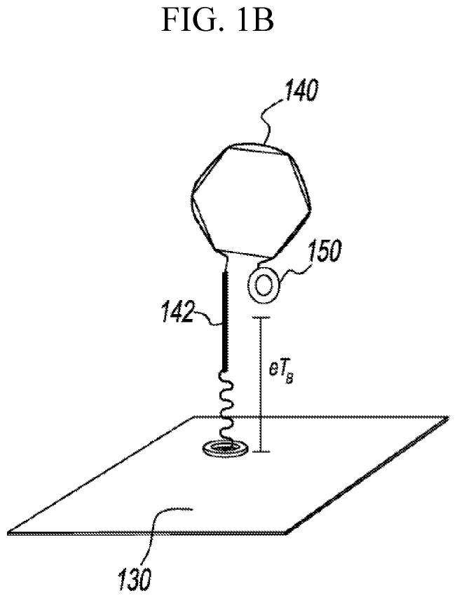

In some embodiments of the device of the present invention, electrochemical aptamer-based (EAB) sensors are used. With reference to FIG. 1A, an aptamer sensing element is depicted. While the figure depicts, and the discussion focuses on, a single aptamer sensing element, EAB sensors described herein will include a large number (thousands, millions, or billions of individual sensing elements, having an upper limit of 1014/cm²) attached to the electrode. The aptamer sensing element 110 includes an analyte capture complex 112, which in turn is comprised of a randomized aptamer sequence 140 that is selected to interact with a target analyte molecule 160, and one or more linker nucleotide sections 142 (one is depicted). The analyte capture complex 112 has a first end covalently bonded to a sulfur molecule, e.g., a thiol 120, which is in turn covalently bonded to an electrode base 130. The electrode 130 may be comprised of gold or another suitable conductive material. The sensing element further includes a redox moiety 150 that may be covalently bonded to a second end of the analyte capture complex 112 or bound to it by a linking section. In the absence of the target analyte, the aptamer 140 is in a first configuration, and the redox moiety 150 is in a first position relative to the electrode 130. When the device interrogates the sensing element using, e.g., square wave voltammetry (SWV), the sensing element produces a first electrical signal, cTA.

With reference to FIG. 1B, the aptamer 140 is selected to specifically interact with a target analyte and one or more linker nucleotide sections 142, so that when the aptamer interacts with a target analyte molecule, the aptamer undergoes a conformation change that partially disrupts the first configuration and forms a second configuration. The capture of the target analyte accordingly moves the redox moiety 150 into a second position relative to the electrode 130. Now when the biofluid sensing device interrogates the sensing element, the sensing element produces a second electrical signal, eTB that is distinguishable from the first electrical signal. After a recovery interval, the aptamer releases the target analyte, and the aptamer will return to the first configuration, which will produce the corresponding first electrical signal when the sensing element is interrogated.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention.

The present invention includes a means to establish fluid communication between the sensor(s) and the sample of interstitial fluid, blood or both. In many embodiments, the means will involve penetrating the dermis to access biofluids such as interstitial fluid and blood. In one embodiment, the means of establishing fluid communication uses a single needle. In another embodiment, the means of establishing fluid communication uses a multitude of microneedles.

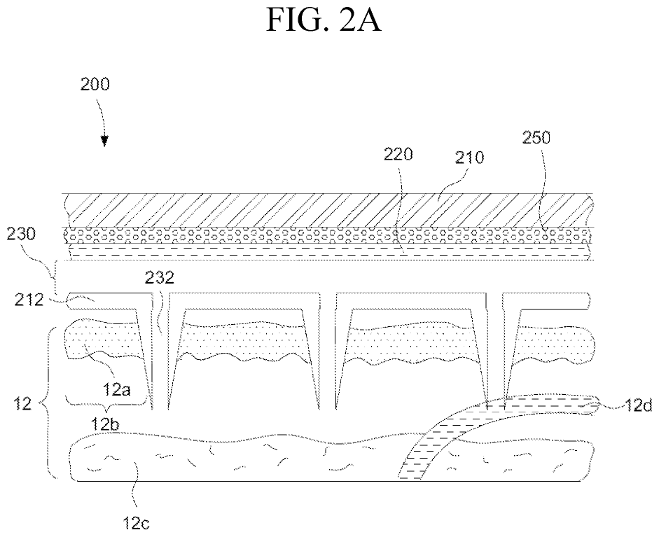

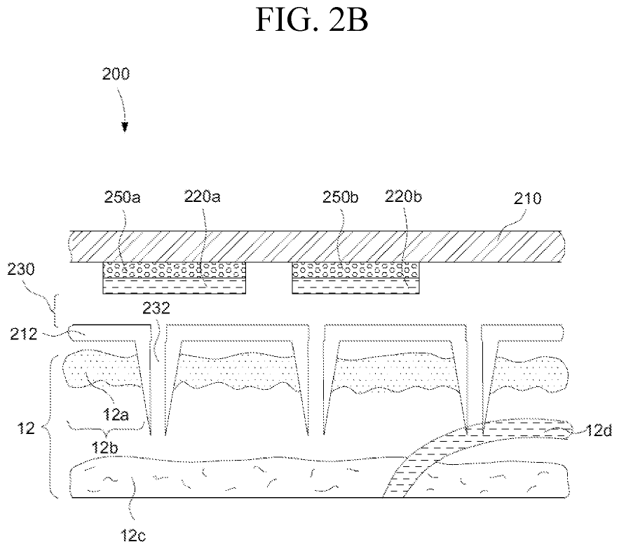

With reference to FIG. 2A, in an embodiment of the disclosed invention, a device 200 is placed partially in-vivo into the skin 12 comprised of the epidermis 12a, dermis 12b, and the subcutaneous or hypodermis 12c. A portion of the device 200 accesses fluids such as interstitial fluid from the dermis 12b and/or blood from a capillary 12d. Access is provided, for example, by microneedles 212 formed of metal, polymer, semiconductor, glass or other suitable material, and may include a hollow lumen 232 that contributes to a sample volume. Sample volume is also contributed to by volume 230 above material from which the microneedles 212 project yet below sensor probes 220 on electrode 250 on a polymer substrate 210. Together, probes 220 and electrode 250 form a sensor 220, 250. Together the volume of volume 230 and lumen 232 form a sample volume and can be a microfluidic component such as channels, a hydrogel, or other suitable material. A diffusion and/or advective flow pathway exists from the invasive biofluid such as interstitial fluid or blood to the sensor probes 220, the pathway beginning at the inlet to the microneedle 212, first reaching the sensor probes 220. Alternative arrangements and materials are possible, such as using a single needle, hydrogel polymer microneedles, or other suitable means to couple an invasive fluid to one or more sensors, although these alternative arrangements and materials are not explicitly shown in the figures. In addition, one or more of the features of device 200 or the entire device 200 may be implanted into the body and perform similarly as described herein.

With further reference to FIG. 2A, sensor probes 220 are affinity-based and, in one embodiment, comprise aptamer sequences that are selective in reversible binding to an analyte and permanently thiol bonded to the electrode 250 and used to sense an analyte such as glucose or other analyte by means of electrochemical detection. In some embodiments, the electrode 250 includes gold. In some embodiments, probes are electrical in nature and utilize an attached redox couple to transduce the electrochemical signal or instead measure change in impedance between the electrode and solution. In other embodiments, probes are optical in nature, such as fluorescently labeled aptamers that are labeled with a quencher (i.e. molecular beacon) that may not require electrode 250 but may use optical sensors and light sources to detect analyte aptamer interactions. Such alternative arrangements are not explicitly shown in the figures.

With reference to FIG. 2B, where like numerals refer to like features, a plurality of sensors for a plurality of analytes are provided. In one embodiment, sensor 250a and sensor 220a detect LH, while sensor 250b and sensor 220b detect progesterone. This figure shows that a device may have one or more sensors for one or more analytes. As in the previous figure, a device 200 is placed partially in-vivo into the skin 12 comprised of the epidermis 12a, dermis 12b, and the subcutaneous or hypodermis 12c. A portion of the device 200 accesses fluids such as interstitial fluid from the dermis 12b and/or blood from a capillary 12d. Access is provided, for example, by microneedles 212 formed of metal, polymer, semiconductor, glass or other suitable material, and may include a hollow lumen 232 that contributes to a sample volume. Sample volume is also contributed to by volume 230 above material from which the microneedles 212 project yet below sensor probes 220a, 220b on electrodes 250a, 250b on a polymer substrate 210.

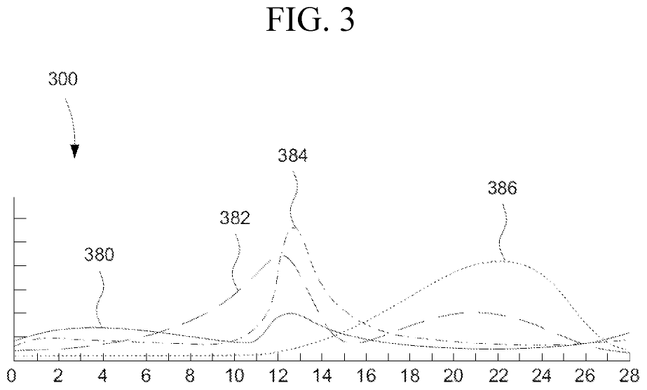

With reference to FIG. 3, an example plot 300 of hormone levels during a woman's ovulation cycle is shown with plot line 380 indicating levels of follicle stimulating hormone (FSH), plot line 382 indicating levels of estrogen, plot line 384 indicating levels of LH, and plot line 386 indicating levels of progesterone. The devices of the present invention may be continuous or for measuring a single concentration of the analyte or analytes. In one embodiment, the devices of the present invention comprise at least one aptamer sensor and at least one needle which couples the aptamer sensor to a biofluid such as interstitial fluid. The present invention may be used to measure any of these analytes and/or their metabolites as well. Several methods of testing are now described, which are superior to current once-a-day urine testing methods.

With reference to embodiments of the present invention, in a first method testing scenario, a urine-based LH strip is used at least twice prior to the expected LH peak. When the LH test strips indicate an increase in LH (surge), which requires at least two such tests, at least one device of the present invention is applied to the skin to measure LH continuously for at least one of 6 hours, 12 hours, 24 hours, 2 days, 3 days, or 6 days.

With reference to embodiments of the present invention, a second method testing scenario further includes using the device of the present invention to measure progesterone. This measure would be for at least one of 6 hours, 12 hours, 24 hours, 2 days, 3 days, or 6 days. The second method may be performed after the first method with a distinct device and sensor, or it may be performed with the same device used in the first method by the device containing sensors for both LH and progesterone.

With reference to embodiments of the present invention, in a third method, one or more devices measure one or more analytes of FSH, LH, progesterone, or estrogen, or their metabolites, for at least one of 6 hours, 12 hours, 24 hours, 2 days, 3 days, 6 days, 2 weeks, or 4 weeks.

In one embodiment, the device of the present invention is single use. In another embodiment, referring again to FIG. 2A, the device is pre-wetted with fluid (such as buffer fluid) in spaces 230, 232. The analytes are coupled to the sensors by diffusion. In an alternative embodiment, the device is dry and fluid flows into spaces 230, 232 by positive pressure of interstitial fluid as the device is pressed against skin or by capillary action that pulls interstitial fluid and/or blood into the device spaces 230, 232.

In one embodiment, the device the present invention reports continuous concentration data for one or more analytes to the user. For example, information may be reported through a wirelessly connected smart phone that displays one or more plots of analyte concentration like that shown in FIG. 3. In another embodiment, the present invention reports to the user simple single data point data such as predicted time of ovulation, predicted time of peak fertility both based on LH, and/or a simple yes/no answer of did the user ovulate based on progesterone measurements.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention

What is claimed is:

1. A device for measuring one or more analytes in a sample of interstitial fluid, blood or both, comprising;
at least one electrochemical aptamer-based (EAB) sensor comprising one or more attached redox couples that measure at least one of said analytes;
a means to establish fluid communication between the at least one sensor and the sample of interstitial fluid, blood or both;
wherein the one or more analytes are selected from the group consisting of progesterone, luteinizing hormone (LH), estrogen, follicle stimulating hormone (FSH), their metabolites, and combinations thereof.

2. The device of claim 1 wherein the means to establish fluid communication comprises at least one needle.

3. The device of claim 2 wherein the at least one needle is hollow and it establishes fluid communication of the sample of interstitial fluid or blood to the at least one sensor by diffusion.

4. The device of claim 2 wherein the at least one needle is hollow and it establishes fluid communication of the sample of interstitial fluid or blood to the at least one sensor by advection.

5. The device of claim 1, wherein the device is capable of continuous measurement of one or more analytes for at least 6 hours.

6. The device of claim 1, wherein the device is capable of continuous measurement of one or more analytes for at least 24 hours.

7. The device of claim 1 wherein the device is capable of taking a single measurement.

8. The device of claim 1, comprising a first EAB sensor and a second EAB sensor, wherein the first EAB sensor measures LH and the second EAB sensor measures progesterone.

9. A method of predicting and/or confirming ovulation of a subject comprising;
applying a device for measuring one or more analytes from interstitial fluid, blood or both of the subject; and
measuring a concentration change of luteinizing hormone (LH), progesterone or both in the subject;
wherein the one or more analytes are selected from the group consisting of progesterone, luteinizing hormone (LH), estrogen, follicle stimulating hormone (FSH), their metabolites, and combinations thereof; and further, wherein the device comprises:
a) at least one electrochemical aptamer-based (EAB) sensor comprising one or more attached redox couples that measure at least one of said analytes; and
b) a means to establish fluid communication between the at least one sensor to interstitial fluid, blood or both.

10. The method of claim 9 further comprising the use of at least two urine-based LH test strips to indicate a time for use of the device, and wherein said device continuously measures at least LH for at least 6 hours.

11. The method of claim 10 wherein said device continuously measures at least LH for at least 24 hours.

12. The method of claim 9, further comprising taking at least one measurement of progesterone by a device containing a sensor for progesterone.

13. The method of claim 9 wherein the device contains sensors for both LH and progesterone and measures them both continuously for at least 6 hours.

14. The method of claim 9 wherein the device provides data selected from the group consisting of continuous concentration data of at least one of said analytes, a predicted time of ovulation, a predicted time of peak fertility, a confirmation that ovulation has occurred, and combinations thereof.
